(19) United States
(12) Patent Application Publication
(10) Pub. No.: US 2026/0165695 A1
(43) Pub. Date: Jun. 18, 2026

(54) LEFT ATRIAL APPENDAGE CLOSURE DEVICES

(71) Applicant: BOSTON SCIENTIFIC SCIMED, INC., MAPLE GROVE, MN (US)

(72) Inventors: Philip Andrew Litecky, Forest Lake, MN (US); Troy Anthony Giese, Blaine, MN (US)

(73) Assignee: BOSTON SCIENTIFIC SCIMED, INC., MAPLE GROVE, MN (US)

(21) Appl. No.: 19/415,048

(22) Filed: Dec. 10, 2025

Related U.S. Application Data
(60) Provisional application No. 63/733,123, filed on Dec. 12, 2024.

(51) Int. Cl. A61B 17/00

(52) U.S. Cl. CPC A61B 17/0057; A61B 2017/00477; A61B 2017/0061; A61B 2017/00632; A61B 2017/00867

(57) ABSTRACT

A left atrial appendage closure (LAAC) device is adapted for occluding a patient's left atrial appendage (LAA). The LAAC device includes an expandable frame that is adapted to expand from a compressed configuration to an expanded configuration at a first expansion rate; and an expandable foam body that is disposed relative to the expandable frame and is adapted expand from a compressed configuration to an expanded configuration at a second expansion rate. The LAAC device is adapted to accommodate differences between the first expansion rate and the second expansion rate.

### CROSS-REFERENCE TO RELATED APPLICATIONS

This application claims the benefit of priority of U.S. Provisional Application No. 63/733,123 filed Dec. 12, 2024, the entire disclosure of which is hereby incorporated by reference.

### TECHNICAL FIELD

The disclosure relates generally to medical devices and more particularly to medical devices that incorporate a shape memory foam component.

### BACKGROUND

Medical devices implanted within the heart may include left atrial appendage closure (LAAC) devices, which are intended to close off the left atrial appendage (LAA) in order to reduce the likelihood of thrombi forming in the LAA from escaping the LAA and entering the bloodstream. Thrombi that migrate through the blood vessels may eventually plug a smaller vessel downstream and thereby contribute to stroke or heart attack. Clinical studies have shown that the majority of blood clots in patients with atrial fibrillation originate in the LAA. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a left atrial appendage closure (LAAC) device that is adapted for occluding a patient's left atrial appendage (LAA). The LAAC device includes an expandable frame that is adapted to expand from a compressed configuration to an expanded configuration at a first expansion rate, and an expandable foam body that is disposed relative to the expandable frame and that is adapted to expand from a compressed configuration to an expanded configuration at a second expansion rate. The LAAC device may be adapted to accommodate differences between the first expansion rate and the second expansion rate.

Alternatively or additionally, the LAAC device may further include one or more attachment features coupling the expandable foam body to the expandable frame.

Alternatively or additionally, the expandable frame may include a plurality of struts, and the one or more attachment features may be adapted to translate along one or more of the plurality of struts.

Alternatively or additionally, the one or more attachment features may be embedded within the expandable foam body.

Alternatively or additionally, the one or more attachment features may contain flexible elements that extend between the expandable frame and the expandable foam body.

Alternatively or additionally, the expandable frame may be adapted to anchor the LAAC device within the LAA.

Alternatively or additionally, the expandable frame may be made of a shape memory cage.

Alternatively or additionally, the expandable foam body may include slots that accommodate the expandable frame when the expandable foam body expands.

Alternatively or additionally, the expandable frame may be made of a shape memory material.

Alternatively or additionally, the expandable foam body may be made of an expandable shape memory polymer foam.

Another example may be found in a left atrial appendage closure (LAAC) device that is adapted for occluding a patient's left atrial appendage (LAA). The LAAC device includes an expandable frame that is adapted to expand at a first expansion rate, an expandable foam body that is disposed relative to the expandable frame and is adapted to expand at a second expansion rate, and one or more attachment features that couple the expandable foam body to the expandable frame.

Alternatively or additionally, the one or more attachment features may be slidingly secured relative to the expandable frame.

Alternatively or additionally, the one or more attachment features may be embedded within the expandable foam body.

Alternatively or additionally, the expandable frame may include a plurality of struts.

Alternatively or additionally, the one or more attachment features may include one or more rings that are slidingly disposed on one or more of the plurality of struts.

Another example may be found in a left atrial appendage closure (LAAC) device that is adapted for occluding a patient's left atrial appendage (LAA). The LAAC device may include an expandable frame that is adapted to expand at a first expansion rate, an expandable foam body that is disposed relative to the expandable frame and is adapted to expand at a second expansion rate, and a plurality of flexible elements that extend between the expandable frame and the expandable foam body.

Alternatively or additionally, the expandable frame may be made of a shape memory cage.

Alternatively or additionally, the expandable frame may be made of an expandable stent.

Alternatively or additionally, the plurality of flexible elements may secure the expandable foam body relative to the expandable frame while allowing relative movement between the expandable foam body and the expandable frame.

Alternatively or additionally, the expandable foam body may include a plurality of anchors that are adapted to engage tissue within the LAA.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage);

FIG. 2 is a schematic view of an illustrative LAAC device shown disposed within an LAA in a compressed configuration;

FIG. 3 is a schematic view of the illustrative LAAC device of FIG. 2, shown with an expandable frame of the LAAC device in an expanded configuration and an expandable foam body of the LAAC device in a compressed configuration;

FIG. 4 is a schematic view of the illustrative LAAC device of FIG. 2, shown with the expandable frame in an expanded configuration and the expandable foam body in an expanded configuration;

FIG. 5 is a perspective view of the expandable frame of the illustrative LAAC device of FIG. 2, including an attachment feature;

FIG. 5A is a perspective view of an attachment feature that may be used with the illustrative LAAC device of FIG. 5;

FIG. 6 is a schematic view of an illustrative LAAC device shown in a compressed configuration;

FIG. 7 is a schematic view of the LAAC device of FIG. 6, shown with an expandable frame of the LAAC device in an expanded configuration and an expandable foam body of the LAAC device in a compressed configuration;

FIG. 8 is a schematic view of the LAAC device of FIG. 6, shown with the expandable frame in an expanded configuration and the expandable foam body in an expanded configuration;

FIG. 9 is a schematic view of an illustrative LAAC device shown in a compressed configuration;

FIG. 10 is a schematic view of the LAAC device of FIG. 9, shown with an expandable frame of the LAAC device in an expanded configuration and an expandable foam body of the LAAC device in a compressed configuration; and

FIG. 11 is a schematic view of the LAAC device of FIG. 9, shown with the expandable frame in an expanded configuration and the expandable foam body in an expanded configuration.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

In some instances, a left atrial appendage closure (LAAC) device is adapted for occluding a patient's left atrial appendage (LAA). The LAAC device includes an expandable frame that is adapted to expand from a compressed configuration to an expanded configuration at a first expansion rate and an expandable foam body that is disposed relative to the expandable frame and is adapted to expand from a compressed configuration to an expanded configuration at a second expansion rate. The LAAC device is adapted to accommodate differences between the first expansion rate and the second expansion rate.

In some cases, the LAAC device may further include one or more attachment features that couple the expandable foam body to the expandable frame. In some cases, the expandable frame may include a plurality of struts, and the one or more attachment features may be adapted to translate along one or more of the plurality of struts. In some cases, the one or more attachment features may be embedded within the expandable foam body. In some cases, the one or more attachment features may include flexible elements that extend between the expandable frame and the expandable foam body. Flexible elements may be adapted to secure the expandable foam body to the expandable frame while allowing for relative movement therebetween. As an example, the flexible elements may be elements that can easily coil and uncoil in order to allow for the expandable foam body expanding more slowly than the expandable frame. As an example, the flexible elements may be tethers or sutures.

In some cases, the expandable frame may be adapted to anchor the LAAC device within the LAA. The expandable frame may include a shape memory cage, for example. In some cases, the expandable foam body may include slots that accommodate the expandable frame when the expandable foam body expands. In some cases, the expandable frame may include a shape memory material. In some cases, the expandable foam body may include an expandable shape memory polymer foam.

In some instances, a left atrial appendage closure (LAAC) device is adapted for occluding a patient's left atrial appendage (LAA). The LAAC device includes an expandable frame that is adapted to expand at a first expansion rate, an expandable foam body that is disposed relative to the expandable frame and is adapted to expand at a second expansion rate, and one or more attachment features that couple the expandable foam body to the expandable frame.

In some cases, the one or more attachment features may be slidingly secured relative to the expandable frame. In some cases, the one or more attachment features may be embedded within the expandable foam body. The expandable frame may include a plurality of struts. The one or more attachment features may include one or more rings that are slidingly disposed on one or more of the plurality of struts.

In some instances, a left atrial appendage closure (LAAC) device is adapted for occluding a patient's left atrial appendage (LAA). The LAAC device includes an expandable frame that is adapted to expand at a first expansion rate, an expandable foam body that is disposed relative to the expandable frame and is adapted to expand at a second expansion rate, and a plurality of flexible elements that extend between the expandable frame and the expandable foam body.

In some cases, the expandable frame may include a shape memory cage. In some cases, the expandable frame may include an expandable stent. In some cases, the plurality of flexible elements may secure the expandable foam body relative to the expandable frame while allowing relative movement between the expandable foam body and the expandable frame. In some cases, the expandable foam body may include a plurality of anchors that are adapted to engage tissue within the LAA.

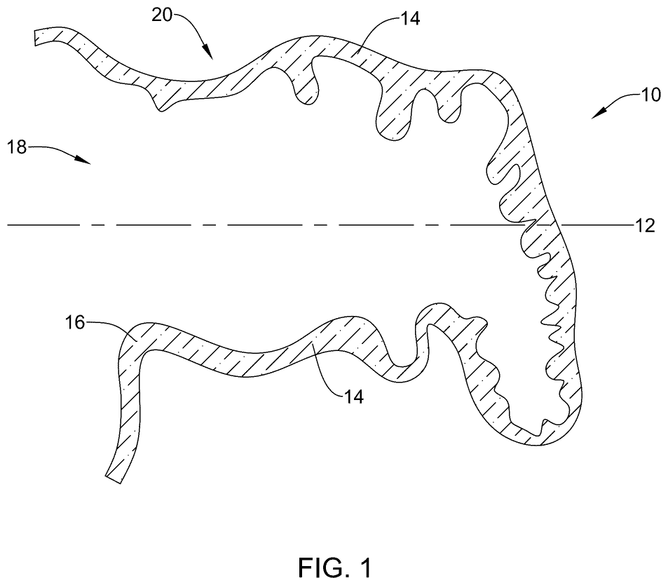

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12. It will be appreciated that FIG. 1 shows an LAA 10 that is just one example of what a left atrial appendage may look like.

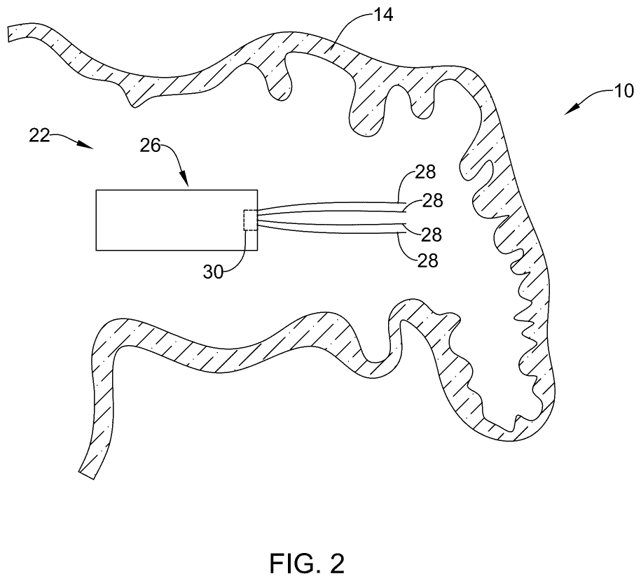

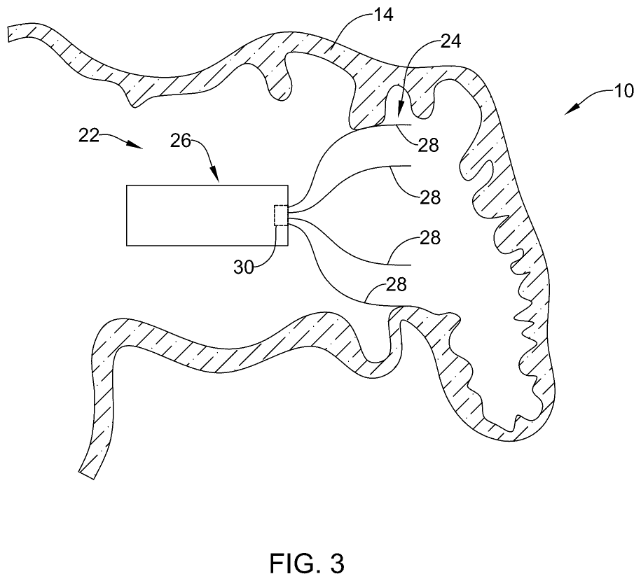

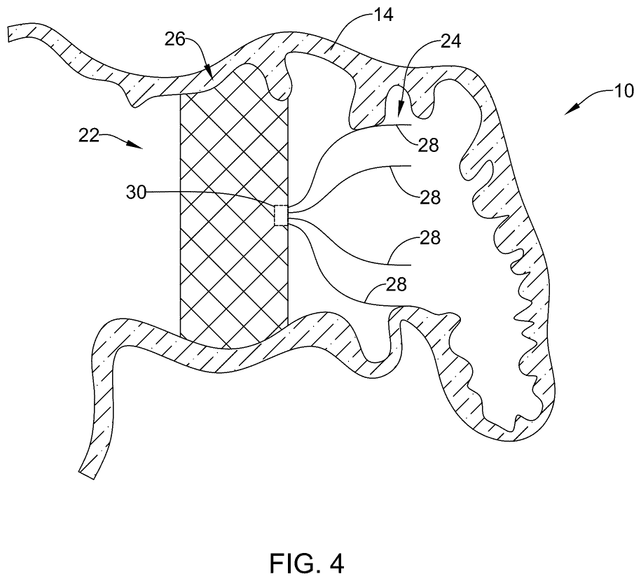

FIG. 2 is a schematic view of an illustrative LAAC device 22 that is shown in a compressed configuration, disposed within the LAA 10. While not shown, at this stage the LAAC device 22 may be coupled with a delivery device. The LAAC device 22 includes an expandable frame 24 that is expandable from a compressed configuration as shown in FIG. 2 and an expandable foam body 26 that is expandable from a compressed configuration as shown in FIG. 2. In FIG. 3, the expandable frame 24 has expanded from its compressed configuration to its expanded configuration while the expandable foam body 26 remains in its compressed configuration. In FIG. 4, the expandable foam body 26 has also expanded into its expanded configuration. In some cases, having the expandable frame 24 and the expandable foam body 26 in their compressed configurations may represent a delivery configuration. After delivery, the expandable frame 24 and the expandable foam body 26 expand into their expanded configurations, which may represent a deployed or implanted configuration.

In some cases, the expandable frame 24 may expand from its compressed configuration to its expanded configuration before the expandable foam body 26 expands from its compressed configuration to its expanded configuration. In some cases, the expandable frame 24 may be considered as expanding at a first expansion rate and the expandable foam body 26 may be considered as expanding at a second expansion rate that is slower than the first expansion rate. In some cases, the expandable frame 24 may expand quickly once delivered while the expandable foam body 26 may expand more slowly. As an example, the expandable frame 24 may expand from its compressed configuration to its expanded configuration as soon as the LAAC device 22 is released from a delivery device (not shown) while the expandable foam body 26 may expand more slowly as the expandable foam body 26 warms and wets in response to blood contact. As an example, the expandable foam body 26 may expand over a period of twenty minutes or longer while the expandable frame 24, particularly if stainless steel or nitinol, may expand instantaneously when deployed.

In some cases, the expandable frame 24 may be formed of a shape memory material. As an example, the expandable frame 24 may be formed of a shape memory metal such as nitinol. The expanded configuration of the expandable frame 24 may represent a remembered shape, and the compressed configuration of the expandable frame 24 may represent a temporary deformation from the remembered shape. In some cases, the expandable frame 24 may expand from its compressed configuration to its expanded configuration as a result of reaching a particular temperature once exposed to body temperature blood or as a result of being released from the containment of the delivery device, for example.

The expandable frame 24 includes a plurality of individual struts 28. When the expandable frame 24 is in its compressed configuration, as shown for example in FIG. 2, each of the individual struts 28 are relatively close together, with each of the individual struts 28 extending largely in an axial direction from the expandable foam body 26. When the expandable frame 24 is in its expanded configuration, as shown for example in FIGS. 3 and 4, each of the individual struts 28 curve outwardly in a radial direction. As can be seen in each of FIGS. 3 and 4, the individual struts 28 curve outwardly a sufficient distance such that at least some of the individual struts 28 make contact with the wall 14 of the LAA 1 (FIG. 1). In some cases, this contact is sufficient to anchor the LAAC device 22 in position within the LAA 10. While not shown, in some cases the individual struts 28 may themselves include hooks or other features that are adapted to further engage with the wall 14 of the LAA 10.

In some cases, the expandable foam body 26 may expand from its compressed configuration to its expanded configuration as a result of being released from a delivery device (not shown). In some cases, the expandable foam body 26 may be formed of a shape memory polymer foam. In some cases, the expandable foam body 26 may expand from its compressed configuration to its expanded configuration as a result of being released from compression. In some cases, the expandable foam body 26 may expand from its compressed configuration to its expanded configuration as a result of the expandable foam body 26 become wetted as a result of exposure to blood and/or warming as a result of being exposed to blood. When the expandable foam body 26 is in its expanded configuration, the expandable foam body 26 may contact the wall 14 of the LAA 10.

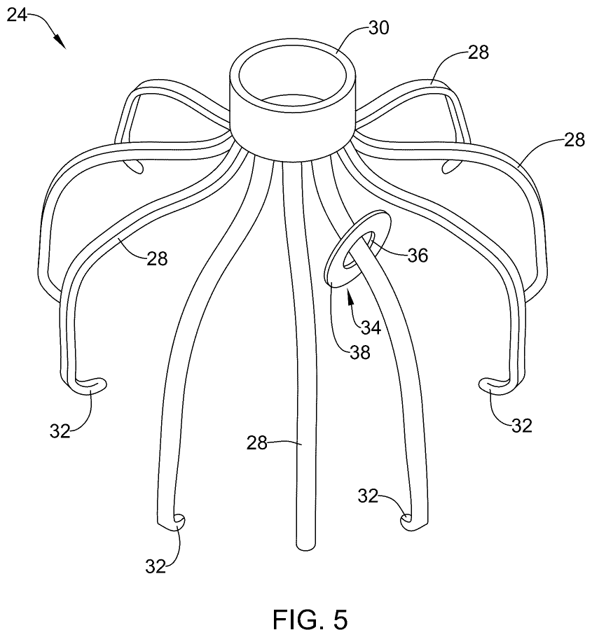

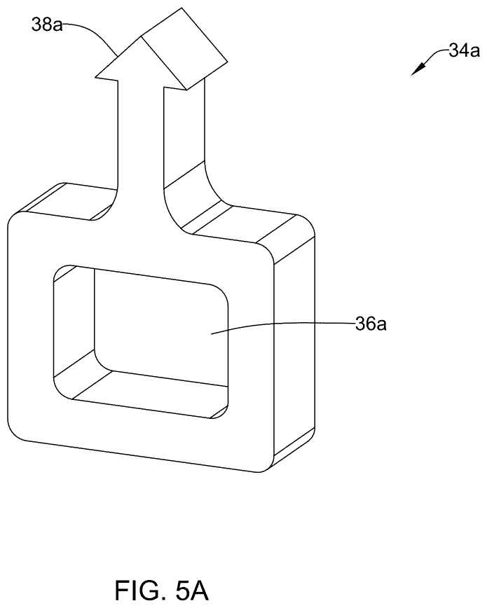

FIG. 5 is a perspective view of the expandable frame 24 that forms part of the LAAC device 22. The expandable frame 24 includes a plurality of struts 28 that each extend away from a central hub 30. In some cases, the central hub 30 may be considered as being embedded within the expandable foam body 26. This can be seen in FIGS. 3 and 4, where the embedded portion of the central hub 30 is shown in dashed line. In some cases, the central hub 30 may be adapted to be releasably secured to a delivery device (not shown). The expandable frame 24 may have any number of individual struts 28. As shown, the expandable frame 24 has a total of nine individual struts 28. In some cases, at least some of the individual struts 28 may have a free end 32 that curves inwardly. In some cases, this may help to make the expandable frame 24 more atraumatic as the LAAC device 22 is advanced into the LAA 10. In some cases, the central hub 30 may be considered as being disposed at a proximal end of the expandable frame 24 while the free ends 32 of each of the individual struts 28 may be considered as being disposed at a distal end of the expandable frame 24.

In some cases, the expandable foam body 26 may extend over at least part of the individual struts 28 forming the expandable frame 24. In some cases, the LAAC device 22 may include one or more attachment features that allow securement between the expandable frame 24 and the expandable foam body 26 while still accounting for differences in when and how each of the expandable frame 24 and the expandable foam body 26 expand when implanted. In some cases, the attachment features may include rings 34 (only one is shown) that are adapted to allow for movement between the expandable frame 24 and the expandable foam body 26. In some cases, there may be a ring 34 slidingly disposed on each of the individual struts 28. In some cases, there may be a ring 34 slidingly disposed on every other of the individual struts 28, for example. Each of the rings 34 includes an aperture 36 that is adapted to slidingly accept one of the individual struts 28 therethrough and an annular surface 38 that is adapted to be embedded within the expandable foam body 26. The rings 34 may be formed of any suitable metal, such as stainless steel or nitinol. While the rings 34 are shown as being circular, this is not required, and the rings 34 may take any desired overall shape as long as each ring 34 is able to translate relative to the individual strut 28 upon which the ring 34 is disposed.

FIG. 5A is a perspective view of an illustrative attachment feature 34a that may be used in place of the rings 34. In some cases, the attachment feature 34a may be used in combination with the rings 34. In some cases, there may be an attachment feature 34a slidingly disposed on each of the individual struts 28. In some cases, there may be an attachment feature 34a slidingly disposed on every other individual strut 28. The attachment feature 34a includes an aperture 36a that is adapted to slidingly accept one of the individual struts 28 therethrough. The attachment feature 34a includes an arrow-shaped portion 38a that is adapted to be embedded within the expandable foam body 26. The attachment features 34a may be formed of any suitable metal, such as stainless steel or nitinol.

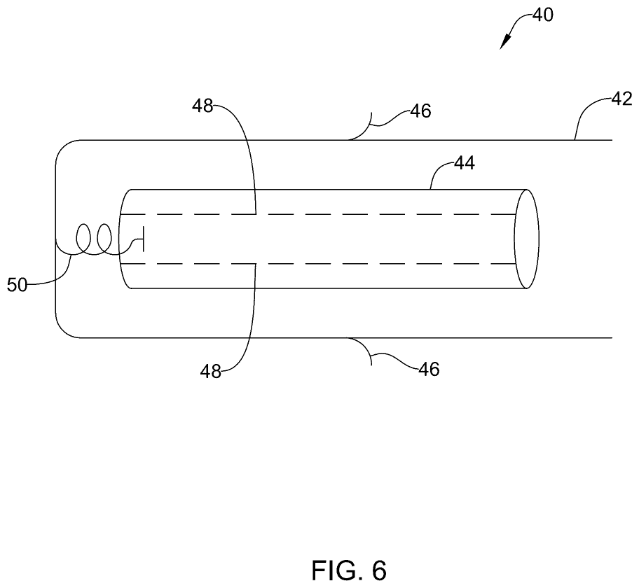

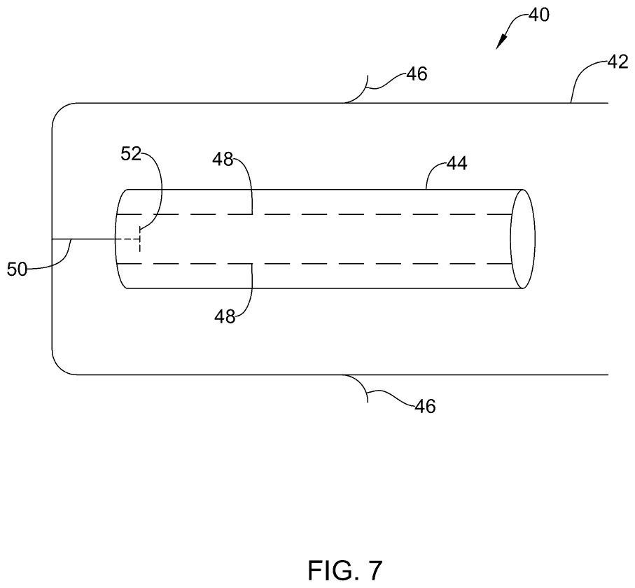

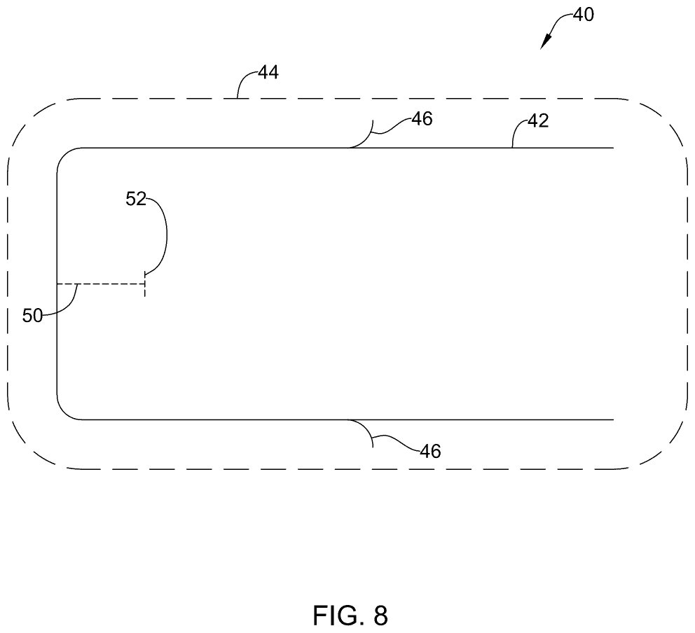

FIG. 6 is a schematic view of an illustrative LAAC device 40 that is shown in a compressed configuration. The LAAC device 40 includes an expandable frame 42 that is expandable from a compressed configuration as shown in FIG. 6 and an expandable foam body 44 that is expandable from a compressed configuration as shown in FIG. 6. In FIG. 7, the expandable frame 42 has expanded from its compressed configuration to its expanded configuration while the expandable foam body 44 remains in its compressed configuration. In FIG. 8, the expandable foam body 44 has also expanded into its expanded configuration. In some cases, having the expandable frame 42 and the expandable foam body 44 in their compressed configurations may represent a delivery configuration. After delivery, the expandable frame 42 and the expandable foam body 44 expand into their expanded configurations, which may represent a deployed or implanted configuration. As an example, the expandable foam body 26 may expand over a period of twenty minutes or longer while the expandable frame 42, particularly if stainless steel or nitinol, may expand instantaneously when deployed.

In some cases, the expandable frame 42 may expand from its compressed configuration to its expanded configuration before the expandable foam body 44 expands from its compressed configuration to its expanded configuration. In some cases, the expandable frame 42 may be considered as expanding at a first expansion rate and the expandable foam body 44 may be considered as expanding at a second expansion rate that is slower than the first expansion rate. In some cases, the expandable frame 42 may expand quickly once delivered while the expandable foam body 44 may expand more slowly. As an example, the expandable frame 42 may expand from its compressed configuration to its expanded configuration as soon as the LAAC device 40 is released from a delivery device (not shown) while the expandable foam body 44 may expand more slowly as the expandable foam body 44 warms and wets in response to blood contact.

In some cases, the expandable frame 42 may be formed of a shape memory material. As an example, the expandable frame 42 may be formed of a shape memory metal such as nitinol. The expanded configuration of the expandable frame 42 may represent a remembered shape, and the compressed configuration of the expandable frame 42 may represent a temporary deformation from the remembered shape. In some cases, the expandable frame 42 may expand from its compressed configuration to its expanded configuration as a result of reaching a particular temperature once exposed to body temperature blood or as a result of being released from the containment of the delivery device, for example. In some cases, the expandable frame 42 may form a cage structure that expands to contact the wall 14 of the LAA 10 (FIG. 1) sufficiently to anchor the LAAC device 40 in place within the LAA 10. In some cases, the expandable frame 42 may include additional features 46 such as hooks or other shapes that further facilitate anchoring the LAAC device 40 in place within the LAA 10.

In some cases, the expandable foam body 44 may expand from its compressed configuration to its expanded configuration as a result of being released from a delivery device (not shown). In some cases, the expandable foam body 44 may be formed of a shape memory polymer foam. In some cases, the expandable foam body 44 may expand from its compressed configuration to its expanded configuration as a result of being released from compression. In some cases, the expandable foam body 44 may expand from its compressed configuration to its expanded configuration as a result of the expandable foam body 44 become wetted as a result of exposure to blood and/or warming as a result of being exposed to blood. When the expandable foam body 44 is in its expanded configuration, the expandable foam body 44 may contact the wall 14 of the LAA 10.

In some cases, the expandable foam body 44 may have an expanded configuration (as seen in FIG. 8) in which the expandable foam body 44 extends radially outwardly farther than the expandable frame 42 extends. In some cases, the expandable foam body 44 may include one or more slits or slots 48 that extend along the expandable foam body 44. The slits or slots 48 form openings in the expandable foam body 44 that allow the expandable foam body 44 to extend around and beyond individual components of the expandable frame 42. While the slits or slots 48 are shown schematically as extending axially along a length of the expandable foam body 44, the expandable foam body 44 may additionally or alternatively include slits or slots 48 that extend circumferentially about the expandable foam body 44.

The LAAC device 40 includes one or more attachment features that secure the expandable foam body 44 relative to the expandable frame 42 while still allowing the expandable frame 42 and the expandable foam body 44 to expand at different expansion rates. As shown, an attachment feature includes a tether 50 that is secured at one end to the expandable frame 42. The other end of the tether 50 includes a portion 52 that is embedded within the expandable foam body 44. The tether 50 may be formed of any suitable material, such as suture material. The tether 50 may be adhesively secured to the expandable frame 42, for example. In some cases, the tether 50 may extend through an aperture (not shown) formed in the expandable frame 42 and may be secured by tying a knot in the tether 50 that is sized so that it cannot be pulled through the aforementioned aperture.

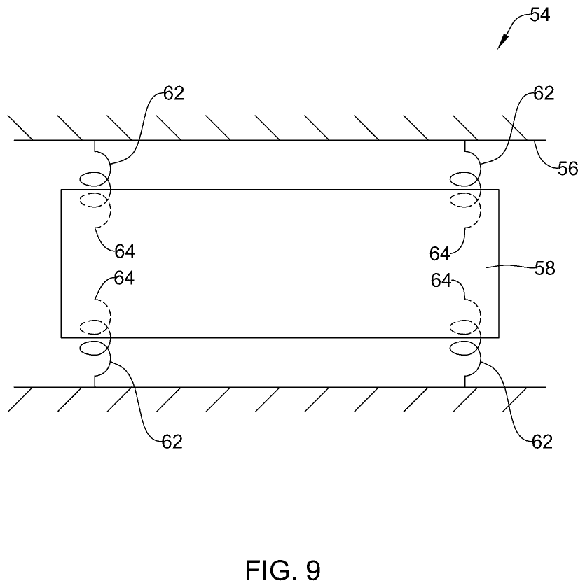

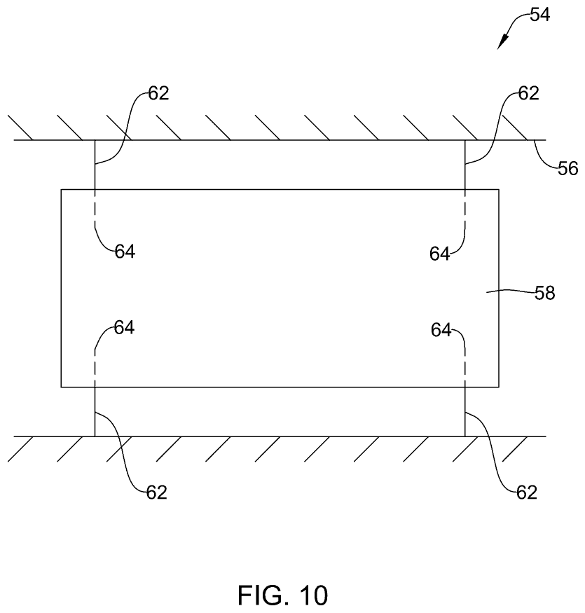

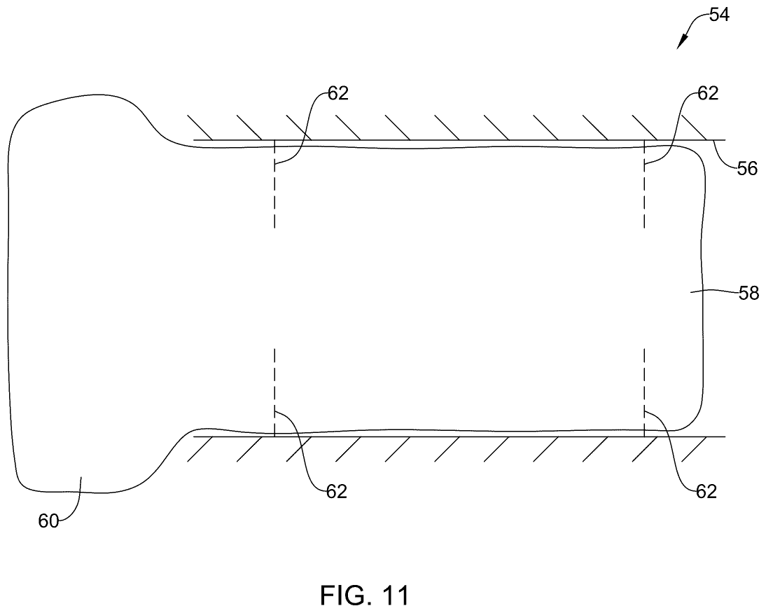

FIG. 9 is a schematic view of an illustrative LAAC device 54 that is shown in a compressed configuration. The LAAC device 54 includes an expandable frame 56 that is expandable from a compressed configuration as shown in FIG. 9 and an expandable foam body 58 that is expandable from a compressed configuration as shown in FIG. 9. In FIG. 10, the expandable frame 56 has expanded from its compressed configuration to its expanded configuration while the expandable foam body 58 remains in its compressed configuration. In FIG. 11, the expandable foam body 58 has also expanded into its expanded configuration. In some cases, having the expandable frame 56 and the expandable foam body 58 in their compressed configurations may represent a delivery configuration. After delivery, the expandable frame 56 and the expandable foam body 58 expand into their expanded configurations, which may represent a deployed or implanted configuration.

In some cases, the expandable frame 56 may expand from its compressed configuration to its expanded configuration before the expandable foam body 58 expands from its compressed configuration to its expanded configuration. In some cases, the expandable frame 56 may be considered as expanding at a first expansion rate and the expandable foam body 58 may be considered as expanding at a second expansion rate that is slower than the first expansion rate. In some cases, the expandable frame 56 may expand quickly once delivered while the expandable foam body 58 may expand more slowly. As an example, the expandable frame 56 may expand from its compressed configuration to its expanded configuration as soon as the LAAC device 65 is released from a delivery device (not shown) while the expandable foam body 58 may expand more slowly as the expandable foam body 58 warms and wets in response to blood contact.

In some cases, the expandable frame 56 may be an expandable stent, which may be a knitted stent, a woven stent or a laser-cut stent, for example. In some cases, the expandable frame 56 may be formed of a shape memory material. As an example, the expandable frame 56 may be formed of a shape memory metal such as nitinol. The expanded configuration of the expandable frame 56 may represent a remembered shape, and the compressed configuration of the expandable frame 56 may represent a temporary deformation from the remembered shape. In some cases, the expandable frame 56 may expand from its compressed configuration to its expanded configuration as a result of reaching a particular temperature once exposed to body temperature blood or as a result of being released from the containment of the delivery device, for example. In some cases, the expandable frame 56 may expand to contact the wall 14 of the LAA 10 (FIG. 1) sufficiently to anchor the LAAC device 54 in place within the LAA 10. In some cases, the expandable frame 56 may include additional features such as hooks or other shapes that further facilitate anchoring the LAAC device 54 in place within the LAA 10.

In some cases, the expandable foam body 58 may expand from its compressed configuration to its expanded configuration as a result of being released from a delivery device (not shown). In some cases, the expandable foam body 58 may be formed of a shape memory polymer foam. In some cases, the expandable foam body 58 may expand from its compressed configuration to its expanded configuration as a result of being released from compression. In some cases, the expandable foam body 58 may expand from its compressed configuration to its expanded configuration as a result of the expandable foam body 58 become wetted as a result of exposure to blood and/or warming as a result of being exposed to blood. When the expandable foam body 58 is in its expanded configuration, the expandable foam body 58 may contact the wall 14 of the LAA 10 through portions of the expandable frame 56. In some cases, a portion of the expandable foam body 58 within the expandable frame 56 may be constrained from expanding radially past the expandable frame 56. In some cases, a proximal portion 60 extending proximally from the expandable foam body 58 is able to extend radially beyond the expandable frame 56 and thus can better seal against the wall 14 of the LAA 10 (FIG. 1).

The LAAC device 54 includes attachment features that secure the expandable foam body 56 relative to the expandable frame 58 while still allowing the expandable frame 56 and the expandable foam body 58 to expand at different expansion rates. As shown, the attachment features include flexible elements (e.g., tethers) 62 that are secured at one end to the expandable frame 56. The other end of each tether 62 includes a portion 64 that is embedded within the expandable foam body 58. Each tether 62 may be formed of any suitable material, such as suture material. Each tether 62 may be adhesively secured to the expandable frame 56, for example. In some cases, each tether 62 may be tied to the expandable frame 56.

The expandable foam may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. In some instances, the expandable foam may be a shape memory foam. Suitable transition temperatures may be, for example, at or below about 37° C. (about 98.6° F.), which allows the shape memory foam to assume an initial configuration prior to and during delivery through a delivery catheter or other delivery device, and an expanded configuration for occlusion after delivery and release within the left atrial appendage, allowing the shape memory foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from about 10 kg/m³ (about 0.62 lb/ft³) to about 1000 kg/m³ (about 62.31 lb/ft³), including from about 10 kg/m³ to about 500 kg/m³ (about 31.2 lb/ft³) including from about 10 kg/m³ to about 200 kg/m³ (about 12.5 lb/ft³), including from about 20 kg/m³ to about 100 kg/m³ (about 6.2 lb/ft³).

Generally, the material for constructing the shape memory foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the shape memory foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the shape memory foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D,L-lactide, poly(beta-hydroxybutyrate), polygluconate including poly-D-gluconate, poly-L-gluconate, poly-D,L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerzation of various diketene acetals and diols; (d) polyanhydride homopolymers and copolymers such as poly(adipic anhydride), poly(suberic anhydride), poly (sebacic anhydride), poly(dodecanedioic anhydride), poly(maleic anhydride), poly[1,3-bis-(p-carboxyphenoxy)methane anhydride], and poly[alpha, omega-bis(p-carboxyphenoxy)alkane anhydride] such as poly[1,3-bis(p-carboxyphenoxy)propane anhydride] and poly[1,3-bis(p-carboxyphenoxy)hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine-and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the shape memory foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others. In some cases, the shape memory polymers may include non-foamed versions of the polymers described herein with respect to making the expandable foams such as shape memory foams. Example of bio-compatible shape memory polymers include polymers made from poly(ε-caprolactone) (PCL), polyurethane (PU), poly (D,L-lactide) (PDLLA), PVA, ethylene vinyl acetate copolymer, (EVA) polymer blend, polymer composites, crosslinked polymers and supramolecular networks, among others. In some instances, shape memory polymers that may be used in creating the foamable solutions described herein may include polyurethane, for example.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A left atrial appendage closure (LAAC) device adapted for occluding a patient's left atrial appendage (LAA), the LAAC device comprising:
an expandable frame adapted to expand from a compressed configuration to an expanded configuration at a first expansion rate; and
an expandable foam body disposed relative to the expandable frame, the expandable foam body adapted to expand from a compressed configuration to an expanded configuration at a second expansion rate;
wherein the LAAC device is adapted to accommodate differences between the first expansion rate and the second expansion rate.

2. The LAAC device of claim 1, further comprising one or more attachment features coupling the expandable foam body to the expandable frame.

3. The LAAC device of claim 2, wherein the expandable frame includes a plurality of struts, and the one or more attachment features are adapted to translate along one or more of the plurality of struts.

4. The LAAC device of claim 3, wherein the one or more attachment features are embedded within the expandable foam body.

5. The LAAC device of claim 2, wherein the one or more attachment features comprise flexible elements that extend between the expandable frame and the expandable foam body.

6. The LAAC device of claim 1, wherein the expandable frame is adapted to anchor the LAAC device within the LAA.

7. The LAAC device of claim 6, wherein the expandable frame comprises a shape memory cage.

8. The LAAC device of claim 6, wherein the expandable foam body includes slots that accommodate the expandable frame when the expandable foam body expands.

9. The LAAC device of claim 1, wherein the expandable frame comprises a shape memory material.

10. The LAAC device of claim 1, wherein the expandable foam body comprises an expandable shape memory polymer foam.

11. A left atrial appendage closure (LAAC) device adapted for occluding a patient's left atrial appendage (LAA), the LAAC device comprising:
an expandable frame adapted to expand at a first expansion rate;
an expandable foam body disposed relative to the expandable frame, the expandable foam body adapted to expand at a second expansion rate; and
one or more attachment features coupling the expandable foam body to the expandable frame.

12. The LAAC device of claim 11, wherein the one or more attachment features are slidingly secured relative to the expandable frame.

13. The LAAC device of claim 11, wherein the one or more attachment features are embedded within the expandable foam body.

14. The LAAC device of claim 11, wherein the expandable frame comprises a plurality of struts.

15. The LAAC device of claim 12, wherein the one or more attachment features comprise one or more rings that are slidingly disposed on one or more of the plurality of struts.

16. A left atrial appendage closure (LAAC) device adapted for occluding a patient's left atrial appendage (LAA), the LAAC device comprising:
an expandable frame adapted to expand at a first expansion rate;
an expandable foam body disposed relative to the expandable frame, the expandable foam body adapted to expand at a second expansion rate; and
a plurality of flexible elements extending between the expandable frame and the expandable foam body.

17. The LAAC device of claim 16, wherein the expandable frame comprises a shape memory cage.

18. The LAAC device of claim 16, wherein the expandable frame comprises an expandable stent.

19. The LAAC device of claim 16, wherein the plurality of flexible elements secure the expandable foam body relative to the expandable frame while allowing relative movement between the expandable foam body and the expandable frame.

20. The LAAC device of claim 16, wherein the expandable foam body comprises a plurality of anchors that are adapted to engage tissue within the LAA.
